(19) United States
(12) Patent Application Publication
(10) Pub. No.: US 2023/0147242 A1
(43) Pub. Date: May 11, 2023

(54) COLORIMETRIC DETECTION OF NUCLEIC ACIDS

(71) Applicant: LIFE TECHNOLOGIES CORPORATION, Carlsbad, CA (US)

(72) Inventors: Alexandre VLASSOV, Simi Valley, CA (US); Xingwang FANG, Austin, TX (US); Madhu JASTI, Austin, TX (US)

(21) Appl. No.: 17/969,419

(22) Filed: Oct. 19, 2022

Related U.S. Application Data
(60) Provisional application No. 63/257,503, filed on Oct. 19, 2021.

(51) Int. Cl. C12Q 1/6844

(52) U.S. Cl. CPC C12Q 1/6846; C12Q 2527/101

(57) ABSTRACT

The present disclosure generally relates to compositions, kits, and methods for the rapid detection of nucleic acid targets in a sample. In some embodiments, a detection reagent comprising at least two metal indicators is disclosed. In additional embodiments, kits and methodologies for detecting the presence or absence of a target nucleic acid sequence comprising the detection reagent comprising multiple metal indicators are provided.

### CROSS-REFERENCE TO RELATED APPLICATIONS

This application claims the benefit of priority to U.S. Provisional Patent Application No. 63/257,503, filed Oct. 19, 2021, which disclosure is herein incorporated by reference in its entirety.

### BACKGROUND OF THE INVENTION

The present disclosure generally relates to the field of nucleic acid detection. More specifically, the disclosure relates to compositions, kits and methods for improving the rapid detection of a nucleic acid target in a sample, including the determination of either the presence or absence of the nucleic acid target using colorimetric detection.

Amplification of nucleic acids, and the capacity for detecting specific nucleic acids of interest, has provided a substantial foundation for the development of molecular biology and related disciplines. One area that has experienced significant development and interest relates to the rapid detection of nucleic acid target sequences associated with emerging and infectious diseases and disorders. In this regard, both thermal cycling dependent process such as a polymerase chain reaction (PCR), as well as isothermal amplification reactions are used to specifically amplify target nucleic acids.

Many current methods for the detection of specific nucleic acid sequences involve fluorescence-based detection. For example, nucleic acid amplification products are often quantified through the use of intercalating fluorophores and for molecular probes (e.g. SYBR™GREEN reagents, ALEXAFLOUR™ reagents, etc.) Fluorescence-based detection methods require specialized equipment for amplification and detection and often require advanced technical or scientific training. Moreover, results usually take hours (or longer) to become available. Fast, reliable, inexpensive, and scalable point-of-care diagnostics that do not require expensive equipment are therefore urgently needed.

### SUMMARY OF THE INVENTION

The instant technology generally relates to compositions, kits and methods for the detection of nucleic acid targets in a sample. A detection reagent for the colorimetric detection of a nucleic acid amplification reaction is provided. The compositions and methods provided herein are based upon the surprising discovery that the spectral properties of an amplification reaction including a combination of two or more metal indicators change when target nucleic acids are amplified, which change can be observed visually and/or with the aid of equipment such as a spectrophotometer.

The detection reagent provided herein is useful across various amplification techniques, an in particular, in isothermal amplification reactions such as a loop-mediated isothermal amplification (LAMP) reaction, a helicase displacement amplification (HPA) reaction, a strand displacement amplification, a recombinase polymerase amplification reaction, a nicking enzyme amplification reaction (NEAR), an exponential amplification reaction (EXPAR), a rolling circle amplification (RCA) reaction and a nucleic acid sequence-based amplification (NASBA) reaction.

As stated above, the detection reagent includes two or more metal indicators. For instance, the detection reagent may include two metal indicators selected from the group consisting of Eriochrome™ Black t, hydroxynaphthol blue (HNB), thymolphthalein complexone, methylthymol blue, xylidyl blue I, xylidyl blue II, calcein, copper sulfate (CuSO₄), calmagite. Preferably, the detection reagent includes HNB and calmagite.

In instances where the detection reagent consists of, or consists essentially of two metal indicators, the molar ratio of the two metal indicators may be between about to about 0.5:4 to about 4:0.5. For example, the molar ratio of the two metal indicators can be between about 0.5:2 to about 2:0.5, including a molar ratio of, e.g. of about 1:1, or 0.75:1.

The detection reagent may be present in a buffer, e.g., an amplification reaction buffer (e.g., for isothermal or thermal amplification), providing a pH in a range of between about 7 and about 10.

Also provided is a kit comprising a detection reagent as provided herein, and one or more components selected from amplification primers, a polymerase, a reaction buffer, and nucleotides. The kits can include more than one polymerase, e.g., 2, 3,4, 5 or more polymerases. For example, the kit includes a reverse transcriptase and a DNA polymerase. For example, the kit can include a Bst or Bsm DNA polymerase (or derivatives thereof) and optionally a reverse transcriptase.

The detection reagent can be provided in the same or separate tubes from one or more of the amplification reaction components. By way of example, the detection can be provided in a tube with the reaction buffer, in a tube with dNTPs, in a tube with the one or more polymerases, in a tube with primers, or the like.

The instant disclosure further provides for a method of detecting the presence or absence of a target nucleic acid in a sample containing nucleic acids comprising a) providing a detection reagent as disclosed herein; b) providing a sample to be tested for the presence or absence of a target nucleic acid; c) providing amplification primers, one or more polymerases, an amplification reaction buffer, and nucleotides; d) generating a reaction mixture comprising the components of a), b) and c); e) subjecting the reaction mixture of d) to amplification conditions; and f) analyzing the spectral properties of the reaction mixture following amplification, wherein an observable change in the optical and/or spectral properties of the reaction mixture indicates the presence of the target nucleic acid(s) in the sample and the lack of discernable change in the observable optical and/or spectral properties of the reaction mixture is indicative of the lack of target nucleic acid(s) in the sample. Step (e) can proceed for a period of time between about 1 minute and about 3 hours, e.g., between about 10 minutes and about 2 hours, e.g., from about 20 min to about 1 hour.

In the methods provided herein, the sample can be a raw sample, e.g., a sample that has not undergone steps to isolate the template nucleic acid(s) from the sample prior to the amplification reaction. Alternatively, the sample can be processed to isolate nucleic acids prior to the amplification reaction.

The detection reagent provided herein may be used in the detection of target nucleic acid sequence(s) derived from a variety of different sample types, including, for example, saliva, nasal swab, nasopharyngeal swab, buccal swab, rectal swab, vaginal swab, sputum, urine, stool, blood, tissue, and semen, environmental samples (e.g., wastewater, sewage, or the like), or any combination thereof (e.g., a nasopharyngeal swab and saliva).

Further aspects of the technology are provided in the instant disclosure, including the detailed description and accompanying examples. However, variations of and changes in these aspects are clearly within the scope of the technology as will be apparent to those skilled in the art based on the disclosure. In addition, throughout the specification and the claims, unless the context expressly requires otherwise, the word “comprise” and its variations, such as “comprises” and “comprising”, will be understood to imply the inclusion of a stated integer, element, or step or group of integers, elements, or steps, but not the exclusion of any additional integer, element, or step or group of integers, elements, or steps.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings are included to further demonstrate certain aspects of the present disclosure.

FIG. 1 is a photograph of a series of loop-mediated isothermal amplification reactions using a single metal detection reagent, as described in Example 1. Control reactions containing amplification primers only (“Primers”) and no template control (“NTC”) were included as indicated. “RNA50”, “RNA100”, and “RNA1000” refer to reactions including 50, 100, and 1000 copies of γ irradiated SARS-CoV-2 viral genome copies, respectively. The reactions included either hydroxynapthol blue (“HNB150 μM”), or calmagite (“Calmagite 150 μM”), as indicated. Following initiation of the reaction, the sample tubes were incubated for 0, 20, 30, 40 or 50 minutes as indicated.

FIG. 2 is a photograph of a series of loop mediated isothermal amplification reactions using different concentrations of a combination of HNB/calmagite as described in Example 2. The reactions labeled “NTC” are negative control (no template). Reactions labeled “25c” included 25 copies of template nucleic acids; “50c” included 50 copies of template nucleic acids; and “100c” included 100 copies of template nucleic acids. The reactions were incubated at 65° C. for the indicated time. The relative concentrations of the two metal indicators are indicated.

FIG. 3 is a photograph of a series of loop-mediated isothermal amplification reactions using a combination of HNB/calmagite as the detection reagent, using various sample types as the sample input, as described in Example 3. Reactions labelled “100c” were spiked with 100 copies of template nucleic acids, and reactions labelled “0c” did not include spiked template nucleic acids (negative control). The sample input was either a saliva sample “Saliva,” or a nasopharyngeal sample “NP,” processed as described in Example 3.

FIG. 4 is a photograph of a series of loop-mediated isothermal amplification reactions using a combination of HNB/calmagite as the detection reagent. The reactions contain various copy numbers of viral genomes of different SARS-CoV-2 strains, as indicated, or no template control (“NTC”). “25c” refers to 25 viral genome copies, “50c” refers to 50 viral genome copies, “100c” refers to 100 viral genome copies, “200c” refers to 200 viral genome copies, “1000c” refers to 1000 viral genome copies, and “10000c” refers to 10000 viral genome copies. Reactions were processed and performed as described in Example 4.

FIG. 5 is a photograph of a series of loop-mediated isothermal amplification reactions using a combination of HNB/calmagite as the detection reagent. The reactions contained various concentrations viral genome copy numbers or concentration of the indicated target nucleic acids (Flu A H2N2, Flu A H1N1, RP2*, RP1*, SARS-CoV-2), no template (“Non-templated controls”), or no virus (“No virus”) as indicated and as described in Example 5. Samples were processed and amplification reactions were performed as described in Example 5.* RPI and RP2 refer to respiratory virus panels, as described in Example 5.

FIG. 6 is an absorbance spectrum of a two LAMP amplification reactions that contained a detection reagent as described herein. One reaction was performed on a sample that was positive for contained SARS-CoV-2 (“SARS-CoV-2 Positive”) and another sample containing was performed on a sample that was negative for SARS-CoV-2 (“SARS-CoV-2 Negative”).

### DETAILED DESCRIPTION OF THE INVENTION

Provided herein are compositions, kits, and methods for the detection of nucleic acid targets in a sample. In particular, the compositions, kits and methods provided herein advantageously allow one to rapidly and accurately identify the presence or absence of a target nucleic acid sequence of interest. The skilled artisan will appreciate that the instant technology beneficially facilitates expedited field testing, reduces testing costs, and eliminates the need for additional components and/or reagents associated, e.g. with more complex nucleic acid targeting and detection assays.

Compositions and Kits

Provided herein is a detection reagent useful for indicating the presence or absence of target nucleic acids in an amplification reaction. The detection reagent provided herein includes two or more metal indicators. A “metal indicator” refers to a compound that exhibits a color change or changes in spectral properties upon binding to one or more metal ions in solution. The detection reagent provided herein can include two or more metal indicators such as Eriochrome™ Black, hydroxynaphthol blue (HNB), thymolphthalein complexone, methylthymol blue, xylidyl Blue I, xylidyl Blue II, calcein, copper sulfate (CuSO₄), calmagite, and combinations thereof. For example, a detection reagent may comprise, consist essentially of, or consist of HNB and calmagite.

In cases where the detection reagent consists of or consists essentially of two metal indicators, the molar ratio of each of the two metal indicators may be between about 0.5:2 to about 2:0.5, including about 0.6:1.8 to about 1.8:0.6, about 0.75:1.5 to about 1.5:0.75, about 0.9:1.1 to about 1.1:0.9 and about 1:1. For example, a detection reagent as described herein may consist of a two-metal indicator system with HNB and calmagite at a molar ratio of about 1:1.

The concentration of the metal indicators in the amplification reaction can be between about 20 μM and about 200 μM, e.g., between about 50 μM and about 150 μM. For example, each detection reagent can be present at a final concentration of about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 175 μM, about 180 μM, about 185 μM, about 190 μM, about 195 μM, about 200 μM, or any concentration in between. The detection reagents provided herein can be provided alone, or for example in combination with one or more reagents for an amplification reaction. The detection reagent can be provided in a concentrated form, in order to achieve the final concentration of the metal indicators in an amplification reaction as indicated above. For example, the detection reagent can be provided in a 2×, 5×, 10×, 20×, 50×, or 100× concentration, to be diluted in an amplification reaction to provide a 1× concentration as described above.

The detection reagents provided herein can be used in numerous amplification techniques, including a thermal cycling dependent process such as a polymerase chain reaction (PCR), and isothermal techniques. Preferably, the detection reagent is used to detect target sequences amplified in isothermal amplification reactions, such as a loop-mediated isothermal amplification (LAMP) reaction, a helicase displacement amplification (HDA) reaction, a strand displacement amplification (SDA) reaction, a recombinase polymerase amplification (RPA) reaction, a nicking enzyme amplification reaction (NEAR), a rolling circle amplification (RCA) reaction and a nucleic acid sequence-based amplification (NASBA) reaction, or the like. For instance, a LAMP reaction may be utilized in conjunction with the detection reagents, kits and/or methodologies described herein. In the presence of target sequences that are amplified, the presence of the target nucleic acid can be detected via a detectable change in the spectral properties of a detection reagent as provided herein (e.g., visually or via an instrument such as a spectrophotometer). In amplification reactions that do not contain target nucleic acids, the absence of a nucleic acid target sequence may be confirmed via, e.g. a lack of detectable change in the spectral properties of the detection reagent provided herein.

The detection reagent can be provided in an amplification reaction buffer. An “amplification reaction buffer,” alternatively referred to as a “reaction buffer,” refers to a buffer capable of sustaining a suitable chemical environment for facilitating nucleic acid sequence amplification during the course of the amplification reaction. The skilled artisan will appreciate that the choice of amplification reaction buffer(s) is often selected based on their capacity for best enhancing the activity of the polymerase operating in the reaction such as, e.g. a Bst DNA polymerase. The amplification reaction buffer(s) may provide a pH level in a range of about 7 to about 10, including a range of about 7.2 to about 9.0, e.g., about 7.5, 7.6, 7.7, 7.8, 7.9, 8.0, 8.1, 8.2, 8.3, 8.4, 8.5 and 8.6, and which may be tailored according to the reaction conditions and/or components, such as the polymerase of choice, for performing an amplification reaction. Preferably, the detection reagents provided herein are used in conjunction with an amplification buffer between a pH of about 8.2 and about 8.8.

The detection reagent can be provided with amplification primers. The term “amplification primers” refers to a oligonucleotides (or analogs) that are capable of initiating the replication of one or more target nucleic acid sequences or complements thereto. Amplification primers useful in the embodiments provided herein include those used in thermal amplification techniques, such as PCR, qPCR, RT PCR, or the like, or for use in isothermal amplification reactions, such as loop-mediated isothermal amplification (LAMP) reactions, helicase displacement amplification (HDA) reactions, strand displacement amplification (SDA) reactions, recombinase polymerase amplification (RPA) reactions, nicking enzyme amplification reactions (NEAR), exponential amplification reactions (EXPAR), rolling circle amplification (RCA) reactions or nucleic acid sequence-based amplification (NASBA) reactions, or the like. Primer design and the quantity of primer(s) included in amplification reactions may be predicated on the type of amplification reaction, the target sequence, etc., using art-accepted methods.

The design of amplification primers useful for isothermal amplification is well-known in the art. For example, in the case of a LAMP reaction, the reaction may include four (4) primers, including a forward outer primer (abbreviated “FOP” or “F3”), a backward outer primer (BOP or B3), a forward inner primer (FIP), and a backward inner primer (BIP). In addition, primers such as a forward loop primer (FLP) and/or a backward loop primer (BLP), may optionally be included, e.g., to accelerate and/or enhance the sensitivity of the LAMP assay.

The detection reagent provided herein can be provided in combination with dNTPs, or nucleotide analogs used in an amplification reaction.

The detection reagent provided herein can be provided in combination with one or more polymerases. For example, the detection reagent provided herein can be provided with a polymerase that has high strand displacement activity and an optimal temperature between about 55° C. and about 70° C. By way of example only, polymerases useful in the compositions, kits and methods provided herein include, but are not limited to Bst DNA polymerase, Bsm polymerase, OMNIAMP™ polymerase (Lucigen, Middleton, Wis.), or the like. In reactions wherein the target nucleic acid is an RNA target, the polymerase preferably has reverse transcriptase activity, or a reverse transcriptase can be included in the reaction. For example, the detection reagent provided herein can be provided with or used in a reaction with a Bsm-derived polymerase and SUPERSCRIPT™ IV reverse transcriptase (Thermo Fisher, cat. 18090010).

The term “kit” as used herein means a set of reagents and/or compositions packaged for carrying out the disclosed methods as described herein. Preferably, the kit comprises one or more of the above-described reagents, for instance amplification primers, one or more polymerases, nucleotides (or appropriate analogs thereof), and a reaction buffer. Optionally, the kits provided herein include instructions for using the detection reagent in an amplification reaction.

Methods

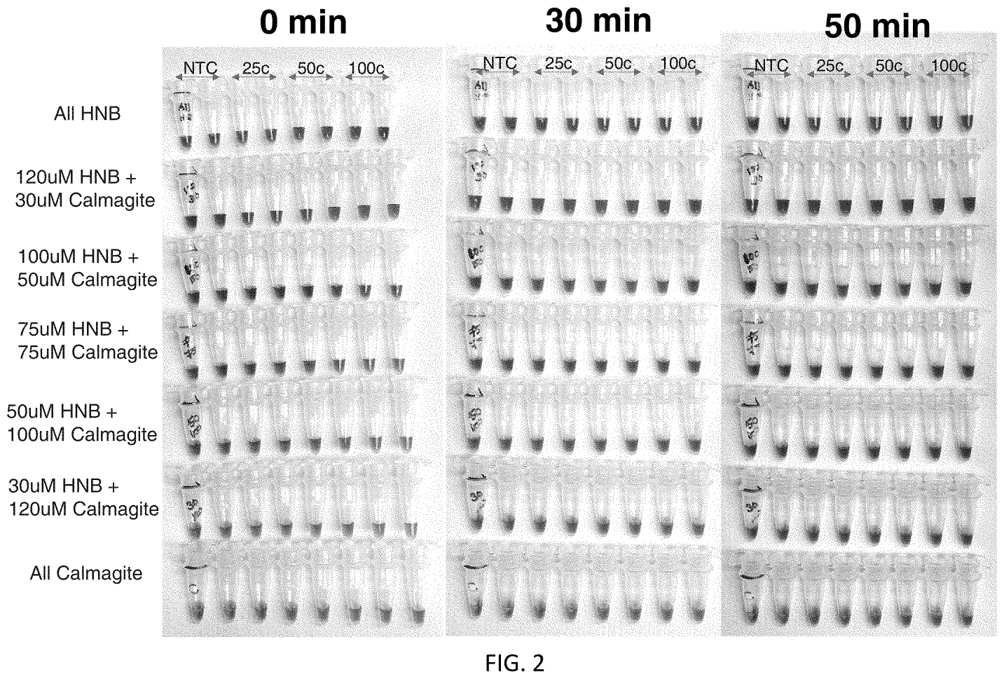

The detection reagent provided herein can be used in amplification reactions, in order to detect the presence or absence of a target nucleic acid (e.g., RNA or DNA), in a sample. Specifically, the presently disclosed detection reagents, as well as the kits and methods associated therewith, beneficially allow for rapid and accurate characterization related to the presence or absence of a nucleic acid target in a biological sample. As shown, e.g. in FIG. 2, the presence or absence of the target nucleic acid sequence can advantageously be discerned in as little as 30 minutes (or less), and does not require, e.g. sending the sample(s) of interest to a separate site or facility for analysis. Moreover, the determination of the presence or absence of the target nucleic acid sequence can be made using relatively inexpensive reagents without the need for costly equipment.

In the methods provided herein, sample nucleic acids are combined with amplification primers specific to a target nucleic acid, dNTPs, an amplification buffer, a detection reagent as provided herein, and one or more polymerases to produce an amplification reaction mixture that is subjected to amplification conditions. Subsequently, the spectral properties of the amplification reaction are analyzed, e.g., visually or spectrophotometrically. The absence of a change in the spectral properties of the detection reagent indicates the absence of target nucleic acids in the sample. The presence of a change in the spectral properties of the detection reagent indicates the presence of target nucleic acids in the sample.

By way of example, a detection reagent that includes HNB and calmagite, e.g., at a ratio between 0.5:1.5, appears purple. In an amplification reaction in which target nucleic acids are not present, the detection reagent remains purple. However, in an amplification reaction in which target nucleic acids are present, the detection reagent appears blue following amplification. (See, FIGS. 2-5). The detection of changes in the spectral properties of the detection reagent provided herein can be achieved by its photochemical properties, e.g., visually without the aid of an instrument, or a spectrophotometer, or the like.

For example, the A650/A540 can be used to determine whether the sample nucleic acids provided in the amplification reaction contained target nucleic acids. As shown in TABLE 1, when using a combination of HNB and calmagite as a detection reagent, the A₆₅₀/A₅₄₀ of the samples that were SARS-CoV-2 negative was lower than 1.0, whereas the A₆₅₀/A₅₄₀ of the samples that were SARS-CoV-2 positive was higher than 1.0.

TABLE 1

| | Plate 1 | | Plate 2 | | Plate 3 | |
|---|---|---|---|---|---|---|
| | Positive | Negative | Positive | Negative | Positive | Negative |
| Attribute | Control | Control | Control | Control | Control | Control |
| A₆₅₀/A₅₄₀ | 1.27 ± 0.0303 | 0.789 ± 0.0517 | 1.01 ± 0.0194 | 0.649 ± 0.0426 | 1.04 ± 0.0189 | 0.684 ± 0.0464 |

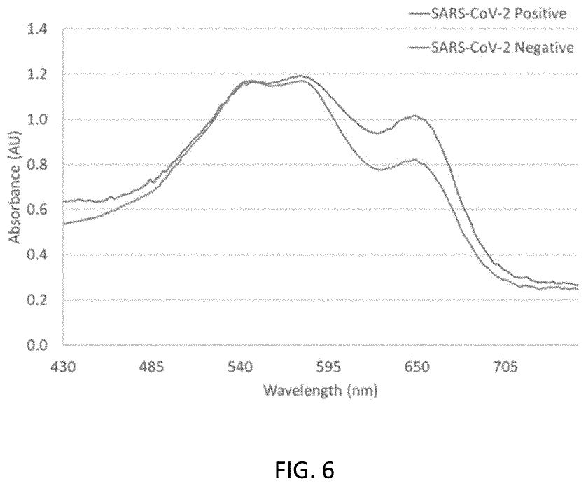

As shown in FIG. 6, whereas the AU₅₄₀ for SARS-CoV-2 Positive and Negative samples were close to the same, the AU₆₅₀ for SARS-CoV-2 Positive samples is much higher than for SARS-CoV-2 Negative samples.

### Examples

The following examples describe improvements related to compositions and methods for the rapid detection of nucleic acid targets in a sample over those previously disclosed in the relevant art. The following examples are provided to demonstrate certain aspects of the disclosed technology.

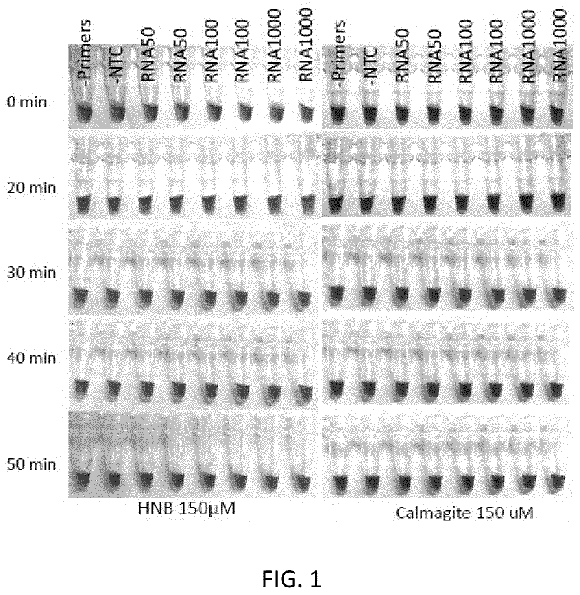

EXAMPLE 1. This example tests the ability of a single metal detection reagent to determine the presence or absence of a target nucleic acid in an isothermal amplification reaction, such as LAMP. Briefly, two different saliva samples were processed using the Applied Biosystems™ MagMAX™ Viral/Pathogen II (MVP II) Nucleic Acid Isolation Kit (Thermo Fisher, cat. A48383), in the KINGFISHER™ Flex Magnetic Particle Processor (Thermo Fisher Scientific, Waltham, Mass.) according to manufacturer's instructions. Each sample was aliquoted into 8 different tubes, including two negative control reactions: “Primers”, which contained primers, and either 150 μM hydroxynapthol blue (HNB 150 μM) or 150 μM calmagite (Calmagite 150 μM); and “NTC” which contained LAMP primers specific for SARS-CoV-2, amplification reaction buffer, dNTPs, and either 150 μM hydroxynapthol blue (HNB 150 μM) or 150 μM calmagite (Calmagite 150 μM). The remaining tubes were spiked with either 50, 100, or 1000 copies of γ-irradiated SARS-CoV-2 (BEI Resources, Cat. No. NR-52287) and contained LAMP primers specific for SARS-CoV-2, amplification reaction buffer, dNTPs. A mixture of Bst-derived DNA polymerase and SUPERSCRIPT™ IV Reverse Transcriptase were added to the reactions, and the reactions were incubated at 65° C. Photographs of the tubes were taken at either 0 min (immediately after addition of the enzyme), 20 min, 30 min, 40 min, or 50 min. The results are shown in FIG. 1.

FIG. 1 shows that the color of LAMP reactions spiked with target nucleic acids (RNA50, RNA100, RNA1000) is not distinguishable from the negative control reactions (Primers, NTC). Thus, these data show that the use of a single metal indicator does not enable visual discrimination between the presence or absence of target nucleic acids in a sample.

EXAMPLE 2: This example illustrates the surprising discovery that the combination of two metal indicators can function as a detection reagent that enables the visual discrimination between the presence or absence of target nucleic acids in an amplification reaction.

Saliva samples were processed using the Applied Biosystems™ MAGMAX™ Viral/Pathogen II (MVP II) Nucleic Acid Isolation Kit (Thermo Fisher, cat. A48383), in the KINGFISHER™ Flex Magnetic Particle Processor (Thermo Fisher Scientific, Waltham, Mass.) according to manufacturer's instructions. The processed samples were aliquoted into 6 different tubes, each containing LAMP primers specific for SARS-CoV-2, reaction buffer, dNTPs, and the indicated concentration of HNB and calmagite. Samples were spiked with water (“NTC”), 25 copies (“25c”), 50 copies (“50c”) or 100 copies (“100c”) of gamma-irradiated SARS-CoV-2 genomes (BEI Resources, Manassas, Va., USA). A mixture of Bst-derived DNA polymerase and SUPERSCRIPT™ IV Reverse Transcriptase was added to the reactions, and the reactions were incubated at 65° C. for 0 minutes, 30 minutes, or 50 minutes as indicated. Photographs of the tubes were taken at the indicated timepoints. The results are shown in FIG. 2.

In contrast to the data on single metal indicators used in FIG. 1, the data shown in FIG. 2 show that the combination of two metal indicators surprisingly enables visual discrimination between amplification reactions that include target nucleic acids (turning blue following amplification), and amplification reactions that do not include target nucleic acids (remaining purple following amplification). This discrimination is achievable across a wide range of relative concentrations of two metal indicators.

EXAMPLE 3: This example illustrates that the detection reagent provided herein can enable visual discrimination of the presence or absence of target nucleic acids in amplification reactions from various types of samples.

Saliva and nasopharyngeal swabs were collected from 12 human donors (D1-D12). Nucleic acids were isolated from the samples using the Applied Biosystems™ MagMAX™ Viral/Pathogen II (MVP II) Nucleic Acid Isolation Kit (Thermo Fisher, cat. A48383) in the KINGFISHER™ Flex Magnetic Particle Processor (Thermo Fisher Scientific, Waltham, Mass.) with 96 Deep-Well Head according to manufacturer's instructions. Aliquots of each processed sample were divided into two reaction tubes containing amplification reaction buffer, dNTPs, and a cocktail of LAMP primers specific for SARS-CoV-2. The samples were spiked with water (“0c”), or 100 copies of γ-irradiated SARSCoV-2 (BEI Resources, Cat. No. NR-52287)(“100c”). The detection reagent in each reaction tube included HNB/calmagite at a molar ratio of about 1:1 (i.e., about 0.075 mM for each of HNB and calmagite). A mixture of Bst-derived DNA polymerase and SUPERSCRIPT™ IV Reverse Transcriptase was added to the reactions, and the reactions were incubated for 0 minutes 30 minutes at 65° C., as indicated.

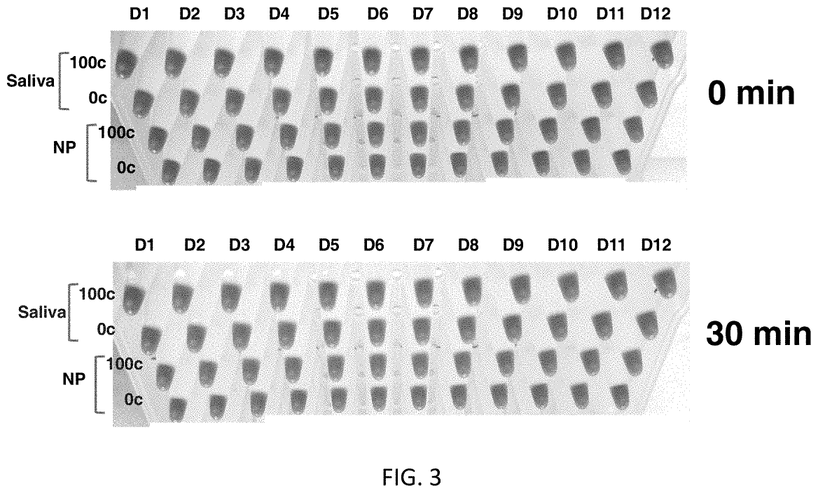

As shown in FIG. 3, an altered colorimetric profile exemplifying either the presence (blue) or absence (purple) of the nucleic acid target sequence was observed after 30 minutes.

EXAMPLE 4: This example illustrates that the detection reagent provided herein can enable visual discrimination of the presence or absence of target nucleic acids from various strains of SARS-CoV-2.

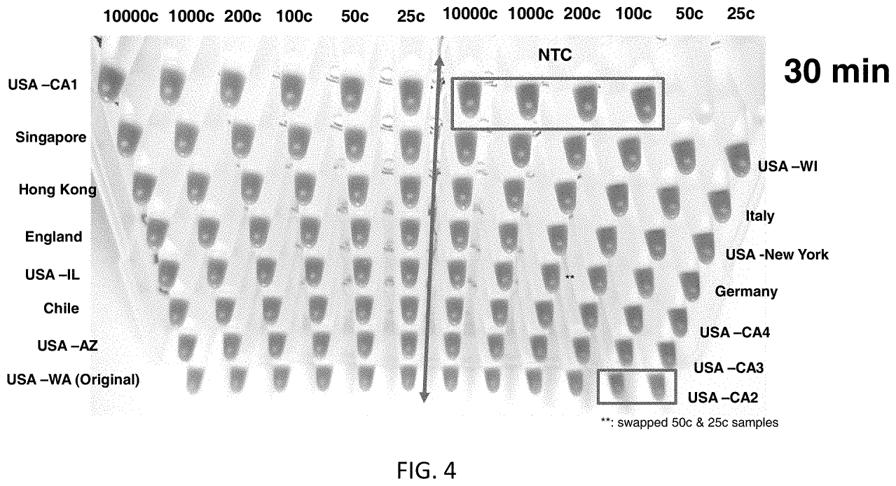

LAMP reactions were set up with 25 copies (“25c”), 50 copies (“50c”), 100 copies (“100c”), 200 copies (“200c”), 1000 copies (“1000c”) or 10,000 copies (“10,000c”) of the following SARS-CoV-2 strains: USA-CA1, Singapore, Hong Kong, England, USA-IL, Chile, USA-AZ, USA-WA, USA-WI, Italy, USA-New York, Germany, USA-CA4, USA-CA3, USA-CA2. The reactions contained dNTPs, amplification buffer, a cocktail of LAMP primers specific for SARS-CoV-2, and a detection reagent (HNB/Calmagite) as described herein. A mixture of Bst-derived DNA polymerase and SUPERSCRIPT™ IV Reverse Transcriptase was added to the reactions, and the reactions were incubated at 65° C. for 30 minutes, and the tubes were subsequently photographed. The results are shown in FIG. 4.

The data from FIG. 4 demonstrate that the detection reagent provided herein is useful in detecting the presence of just 25 copies of target nucleic acids in a sample, and is capable of detecting the presence of different variants of the source of the target nucleic acids when used in a LAMP reaction.

EXAMPLE 5: The following example shows that the detection reagent provided herein can be used to discriminate between samples that contain target nucleic acids, and samples that do not contain target nucleic acids, when used in a LAMP reaction.

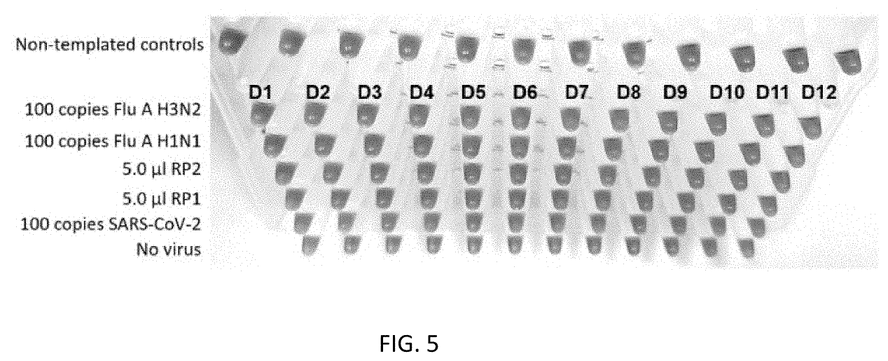

Nucleic acids from samples from 12 unique nasopharyngeal donors (D1-D12) were purified using the MAGMAX™ Viral/Pathogen II Nucleic Acid Isolation kit (Thermo Fisher, cat. A48383) and the KINGFISHER™ Flex Magnetic Particle Processor, according to manufacturer's protocols. Six portions of each sample were aliquoted into tubes, and supplemented with (1) 100 copies of gamma-irradiated SARS-CoV-2 isolate USA-WA1/2020 (BEI Resources), (2) 100 copies of Influenza A H1N1, or (3) 100 copies Influenza A H3N2 (ATCC); (4) 5.0 μl NATTROL™ Respiratory Pathogen Panel-1 (RP-1) (Zeptometrix); (5) or 5.0 μl NATTROL™ Respiratory Pathogen Panel-2 (RP2) (Zeptometrix), or no water (“no virus”). In each tube, 25 μl LAMP reactions were prepared that each contained about 5 μl nucleic acids (or, alternatively, 100 copies of supplemental virus) and a cocktail of LAMP amplification primers specific for SARS-CoV-2. The reactions were allowed to proceed at 65° C. for 30 minutes, at which time they were photographed (FIG. 5).

As shown in FIG. 5, in all samples supplemented with SARS-CoV-2, a positive blue color change was observed, while all other virally supplemented samples and controls remained a negative purple color.

Unless defined otherwise, all technical and scientific terms used herein have the same meaning as is commonly understood by one of ordinary skill in the art(s) to which the embodiments and concepts of the present disclosure belong. While the instant disclosure provides certain illustrative aspects and describes the general principles of the described technology, those persons of ordinary skill in the relevant arts will appreciate that modifications in the arrangement and details of the disclosure may be introduced without departing from these aspects and principles. Accordingly, Applicant claims all modifications that are within the spirit and scope of the appended claims.

1. A detection reagent for colorimetric detection of a nucleic acid amplification reaction, comprising two or more metal indicators.

2. The detection reagent of claim 1, wherein the two or more metal indicators are selected from the group consisting of: Eriochrome™ Black t, hydroxynaphthol blue (HNB), thymolphthalein complexone, methylthymol blue, xylidyl Blue I, xylidyl Blue II, calcein, copper sulfate (CuSO₄) and calmagite.

3. The detection reagent of claim 2, wherein the two or more metal indicators comprise HNB and calmagite.

4. The detection reagent of claim 3, wherein the two or more metal indicators consist essentially of HNB and calmagite.

5. The detection reagent of claim 4, wherein the molar ratio of HNB to calmagite is between about 0.5:2 to about 2:0.5.

6. The detection reagent of claim 5, wherein the molar ratio of HNB to calmagite is about 1:1.

7. The detection reagent of claim 1, wherein the amplification reaction is an isothermal amplification reaction selected from the group consisting of: a loop-mediated isothermal amplification (LAMP) reaction, a helicase displacement amplification (HPA) reaction, a strand displacement amplification, a recombinase polymerase amplification reaction, a nicking enzyme amplification reaction (NEAR), an exponential amplification reaction (EXPAR), a rolling circle amplification (RCA) reaction and a nucleic acid sequence-based amplification (NASBA) reaction.

8. The detection reagent of claim 1, wherein the amplification reaction is loop-mediated isothermal amplification (LAMP).

9. A nucleic acid amplification buffer comprising the detection reagent of claim 1.

10. A nucleic acid amplification reaction comprising the detection reagent of claim 1.

11. The nucleic acid amplification buffer of claim 9, wherein the buffer provides a pH between about 7 and about 10 in the nucleic acid amplification reaction.

12. The nucleic acid amplification reaction of claim 10, wherein the amplification reaction has a pH of between about 7 and about 10.

13. The nucleic acid amplification reaction of claim 10, further comprising one or more reagents selected from the group consisting of: one or more amplification primers, one or more polymerases, one or more buffers, and one or more dNTPs.

14. A kit comprising the detection reagent of claim 1, or the reaction buffer of claim 9.

15. The kit of claim 11, further comprising one or more reagents selected from the group consisting of: one or more amplification primers, one or more polymerases, one or more buffers, and one or more dNTPs.

16. A method of detecting the presence or absence of a target nucleic acid in a sample comprising nucleic acids (sample nucleic acids), comprising:
(a) providing a detection reagent according to claim 1;
(b) providing the sample nucleic acids;
(c) providing amplification primers, one or more polymerases, and dNTPs
(d) generating a reaction mixture comprising a), b) and c);
(e) subjecting the reaction mixture of d) to amplification conditions; and
detecting the optical and/or spectral properties of the detection reagent, wherein the optical and/or spectral properties of the detection reagent change in the presence of the target nucleic acid.

17. The method of claim 16, wherein step e) is allowed to proceed for a period of time between about 10 minutes and about 90 minutes.

18. The method of claim 16, wherein the amplification reaction is an isothermal amplification reaction selected from the group consisting of a loop-mediated isothermal amplification (LAMP) reaction, a helicase displacement amplification (HPA) reaction, a strand displacement amplification, a recombinase polymerase amplification reaction, a nicking enzyme amplification reaction (NEAR), an exponential amplification reaction (EXPAR), a rolling circle amplification (RCA) reaction and a nucleic acid sequence-based amplification (NASBA) reaction.

19. The method of claim 18, wherein the isothermal amplification reaction is LAMP.

20. The method of claim 16, wherein the target nucleic acid is derived from a biological sample selected from the group consisting of saliva, tissue, sputum, urine, blood and semen.
